(19) United States
(12) Patent Application Publication
(10) Pub. No.: US 2005/0033198 A1
(43) Pub. Date: Feb. 10, 2005

(54) Device for assistance in the analysis of adventitious sounds

(76) Inventors: Georges Kehyayan, Clamart (FR); Ivan Kehayoff, Strasbourg (FR)

Correspondence Address: CHRISTIE, PARKER & HALE, LLP, PO BOX 7068, PASADENA, CA, 91109-7068, US

(21) Appl. No.: 10/475,231

(22) PCT Filed: Apr. 16, 2002
(86) PCT No.: PCT/FR02/01307

(30) Foreign Application Priority Data
Apr. 18, 2001 (FR) 01/05256

(51) Int. Cl. A61B 7/00; A61B 5/08

(52) U.S. Cl. 600/586; 600/529

(57) ABSTRACT

The invention concerns a device for assistance in the analysis of adventitious sounds, such as wheezing or crackling sounds. The invention is characterised in that said adventitious sounds are located on time/frequency spectral representations and parameterised according to a set of coefficients characterising them. Said sets of coefficients are stored in a storage unit for establishing a database or compared to synthesized sounds, based on parameters retrieved from such a database.

The invention relates to a device for processing respiratory sounds, such as adventitious sounds of the type comprising sibilant rhonchi, coarse crackles, fine crackles, etc.

Published international application WO 00/00736, to be considered as cited by reference in the present application, discloses a device for processing respiratory sounds of this type, for the purpose of representing, in time and in frequency, a signal representing this sound. As emphasized in the aforementioned international application, this conversion allows a respiratory sound to be “displayed”.

However, the interpretation of the time-frequency spectra obtained is only suitable for technically qualified personnel.

One of the objects of the invention is to improve the device disclosed in application WO 00/00736, in particular its interface with eventual users, especially medical personnel, who alone are competent in diagnosis.

This modification requires in particular for the device to be automated in order to provide a simple and rapid diagnostic aid suitable for use in emergencies.

The invention provides for this purpose a device for assistance in the analysis of respiratory sounds, of the type comprising a working memory, capable of storing samples of a signal representing a respiratory sound, and a computing module capable of cooperating with the working memory in order to carry out a time and frequency conversion of the signal.

According to a general feature of the invention, the device furthermore includes means for identifying, in a converted signal, at least one component relating to an adventitious sound and the computing module is designed to extract a set of parameters characterizing this adventitious sound from the converted signal.

Advantageously, said means are designed to identify the component that represents the adventitious sound in a frequency and time representation of the converted signal.

In one embodiment, the device includes an acquisition member, whereas the means for identifying the component are manual.

Complementarily or as a variant, the computing module is furthermore capable of comparing successive intensity values of the converted signal in order to identify said component.

In this embodiment, the computing module is designed to compare the intensity values by carrying out:
a frequency scan of the intensities, for various successive instants, when the adventitious sound to be identified is a set of sibilant rhonchi; and/or
a time scan of the intensities, for various frequencies, when the adventitious sound to be identified is a set of crackles.

Preferably, the parameters characterizing a set of crackles comprise the number of crackles identified, and also a temporal segment and a mean intensity in this temporal segment, for each crackle.

Provision is furthermore made for these parameters to also include a frequency range within which each identified crackle lies.

Moreover, the parameters characterizing a set of sibilant rhonchi preferably comprise, for each sibilant rhonchus, a mean frequency over a stable phase of the sibilant rhonchus and at least one coefficient of linear variation in frequency of the start and/or of the end of the sibilant rhonchus.

Advantageously, the parameters characterizing a set of sibilant rhonchi comprise the number of fundamental sibilant rhonchi of lowest frequencies.

Particularly to characterize polyphonic sibilant rhonchi, the parameters characterizing a set of sibilant rhonchi comprise, for each fundamental sibilant rhonchus and its harmonic sibilant rhonchi, the number of harmonic sibilant rhonchi and a mean ratio of the intensities of the harmonics to the intensities of the fundamental.

In a preferred embodiment, the computing module is furthermore designed to compare said parameters with values prerecorded in an ordered memory, comprising data of sets of parameters, each set characterizing at least one adventitious sound, the device furthermore including a warning module suitable for being activated or not, depending on the comparison.

In a variant of this embodiment, the parameters characterizing an adventitious sound are stored in succession in an ordered memory, this ordered memory preferably being structured as a database.

In an advantageous embodiment, the device furthermore includes a telecommunication module for remote consultation of the database.

The computing module, for example stored in a read-only memory of the device, is intended to be used by a processor that the device according to the invention includes, as the case may be. It may also be in the form of a computer program that may be loaded into the memory of the device from a removable reader.

In this regard, the object of the present invention is also to provide a removable medium intended to cooperate with a reader of a device of the type described above and comprising data for programs for using the computing module.

In an advantageous embodiment, the removable medium furthermore includes prerecorded data of parameters characterizing adventitious sounds.

Other advantages and features of the present invention will become apparent on examining the detailed description below and the appended drawings in which:

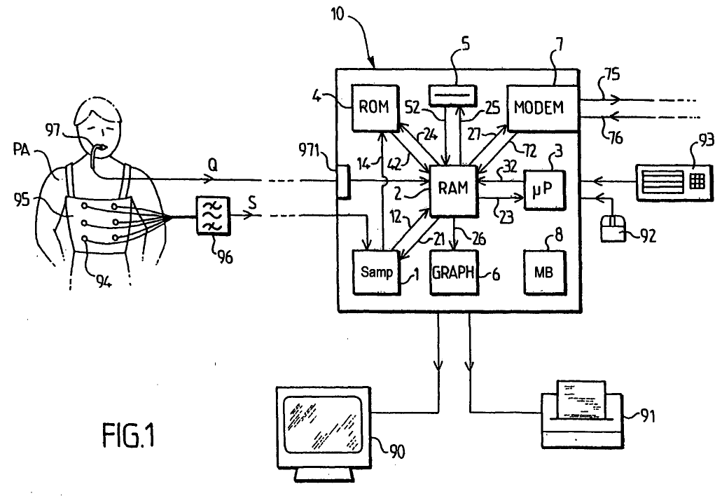

FIG. 1 shows schematically a device according to the invention for analyzing sounds resulting from a mediate auscultation;

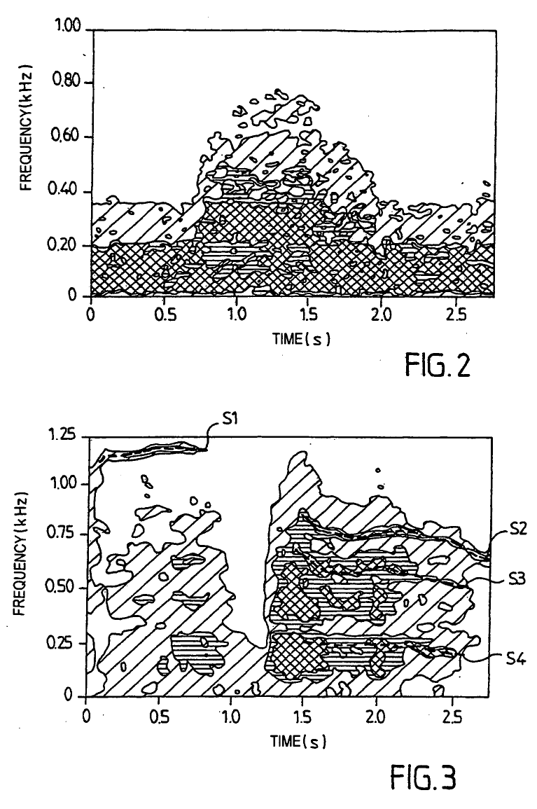

FIG. 2 is a time-frequency representation of a normal respiratory sound (or “vesicular murmur”);

FIG. 3 is a time-frequency representation of a respiratory sound that includes an adventitious sound of the “sibilant rhonchus” type;

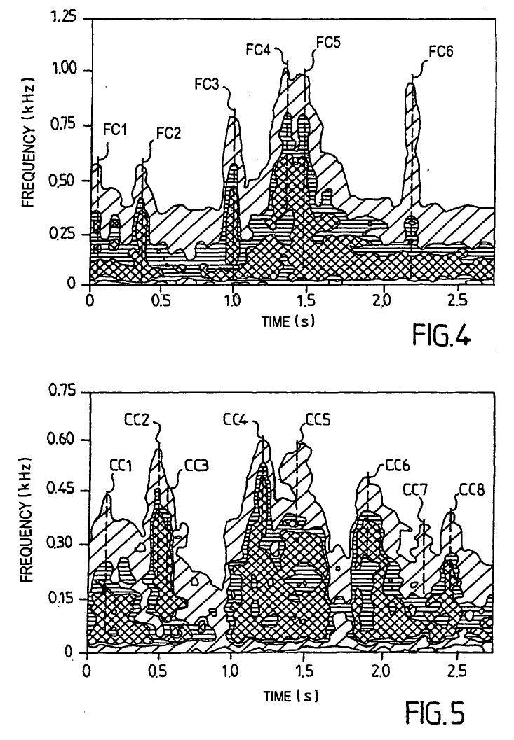

FIG. 4 is a time-frequency representation of a respiratory sound that includes an adventitious sound of the “fine crackle” type; and

FIG. 5 is a time-frequency representation of a respiratory sound that includes an adventitious sound of the “coarse crackle” type.

The drawings contain for the most part elements of a specific nature. They may not only serve to make the present invention more clearly understood, but also, as the case may be, contribute to its definition.

The reader should firstly refer to FIG. 1 which shows, by way of example, a device for analyzing respiratory sounds according to the invention. Overall, this device comprises a processing unit (central processing unit 10), provided with peripherals 90 to 97 that are connected to the central processing unit. In the example shown in FIG. 1, the analytical device includes a computer equipped with a monitor (screen 90), a printer 91, an input keyboard 93 and a screen selection member (or mouse 92).

Moreover, a harness 95 worn by the patient PA includes an electronic stethoscope fitted with a plurality of sensors 94 (six sensors in the example described). These sensors are designed to work in a chosen frequency band so as to avoid picking up sounds emanating from the heartbeats of the patient PA, and also any rubbing noise from the harness 95, or the like. Typically, this frequency band extends from 100 Hz to about 2 kHz. The band-pass filter 96 symbolizes this function. It should be noted that, as a variant, the sensors 94 may work over a wider frequency band, while the link between the sensors 94 and the central processing unit 10, or else the central processing unit itself, include means for filtering within a limited frequency band, between approximately 100 Hz and 2 kHz. The acoustic sensors 94 are then designed to convert an acoustic sound (respiratory sound) into a temporal electrical signal (arrow S).

A respiratory flow rate probe 97 (pneumotachograph in the example described) may also be provided, this being fitted for example into a mask placed on the face of the patient PA. The probe 97 is therefore capable of identifying a start-of-respiration instant, for example between an inspiration and an expiration by the patient PA, from a change in sign of the respiratory flow rate Q.

The central processing unit 10 includes an interface 1 provided with a sampler Samp (present for example on a sound card) connected to the sensors 94, for the purpose of sampling the temporal signal S. In particular, according to Shannon's theorem, it is preferable to use a sampling frequency that is twice the maximum frequency of the useful signal, so as to suppress aliasing phenomena. The samples of the signal S (the aforementioned multiplicity of intensities) are then stored in the random-access memory 2 (RAM), with a view to processing them by a processing module of the aforementioned type, said module being recorded in the read-only memory 4 (ROM) contained in the central processing unit 10.

The central processing unit 10 is provided with a microprocessor 3 (μP) for processing the samples by computation. This microprocessor is advantageously capable of working at computational rates of the order of 150 MHz or higher. The spectra resulting from the processing may be recorded on a hard disk of the central processing unit 10 that includes the read-only memory 4, or else on a removable medium 5, such as a floppy disk, a writable CD-ROM, a magnetooptic disk or the like. There may also be a communication interface 7 with a MODEM link in order to transmit the contents of the random-access memory 2 (processed spectrum) to a remote station, such as a station (not shown) for interpreting data. The communication interface 7 may furthermore receive samples of temporal signals S coming from the digitization of respiratory sounds from a remote patient (not shown) or else already processed spectra, for interpretation.

The link 72 between the modem 7 and the random-access memory 2 of the central processing unit 10, or else the link 12 between the sound card 1 and the random-access memory 2, then form, in this example, the input of the aforementioned analytical device.

The monitor 90 and the printer 91, that the analytical device also has, allow the spectra resulting from the aforementioned processing to be displayed. The input keyboard 93 and the mouse 92 are used to modify the time and frequency scales of the spectra shown, or else to refine the choice of the aforementioned weighting coefficients.

In general, most of the links between the various elements of the central processing unit 10, and the links with the peripherals, are controlled by a motherboard 8 (MB). For the sake of clarity of FIG. 1, the links between the motherboard 8 and the elements of the central processing unit 110 have not been shown. However, it should be noted that the monitor 90 is connected to the central processing unit 10 via a graphics card 6 (link 26) in the usual manner.

The sound card 1 is driven so as to receive a temporal signal that it samples. The read-only memory comprises a computing module so that the processor, in cooperation with the read-only memory, is designed to evaluate a time-frequency transform of the sampled signal, this transform being of the type described in application WO 00/00736.

Accordingly, this application WO 00/00736 is, for all useful purposes, cited here by reference.

Referring now to FIG. 2, this shows the spectrum resulting from such a “time-frequency” conversion of a normal vesicular murmur (nonpathological respiratory sound). The various types of hatching (oblique hatching, horizontal hatching and cross hatching) are representative of ranges of converted (respectively increasing) intensities as a function of a real time scale for which the temporal values are retained, plotted on the x-axis and a frequency scale, plotted on the y-axis. No particular spectral event is therefore noted.

In contrast, the “time-frequency” spectrum shown in FIG. 3 demonstrates substantially four sibilant rhonchi that are continuous sounds having a duration generally greater than or equal to 125 ms:
a first curve S1 from 0 to 0.7 s approximately, with a stabilized frequency of around 1.15 kHz;
a second curve S2 between 1.4 s and 2.7 s, with a mean frequency of around 0.75 kHz;
a third curve S3 that extends from about 1.6 s to 2.5 s and with a mean frequency of around 0.55 kHz; and
a fourth curve S4 between 2 s and 2.5 s, with a mean frequency of around 0.20 kHz.

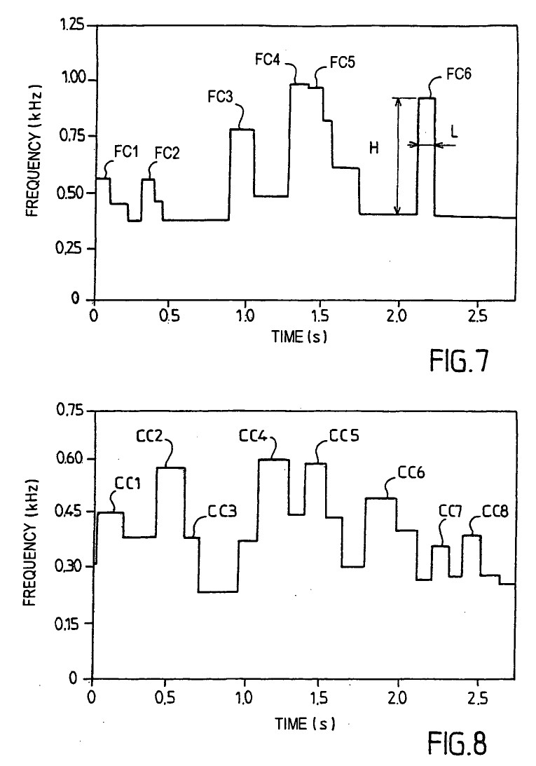

This type of rale is divided into two classes according to whether they are formed from one (monophonic) or several (polyphonic) frequencies. Thus, between 0 and 0.7 s (left-hand part of the spectrum in FIG. 7), the spectral representation of the respiratory sound demonstrates a monophonic sibilant rhonchus, whereas between 1.3 s and 2.5 s, this spectral representation demonstrates a polyphonic sibilant rhonchus (with three frequencies in the spectrum shown).

Another type of adventitious noise (or rale) relates to crackles, which are discontinuous adventitious sounds. The duration of these sounds in general barely exceeds 20 ms.

FIGS. 4 and 5 show fine crackles (FIG. 4) and coarse crackles (FIG. 5). In FIG. 4, pulses FC1 to FC6, of approximately vertical tendency, with a maximum frequency in the region of 1 kHz may be seen. These temporally brief events correspond to fine crackles. In FIG. 5, pulses CC1 to CC8, with frequencies limited to about 600 Hz and substantially longer temporal duration (around 20 ms) may be seen. To the ear, these pulses typically sound like crackles.

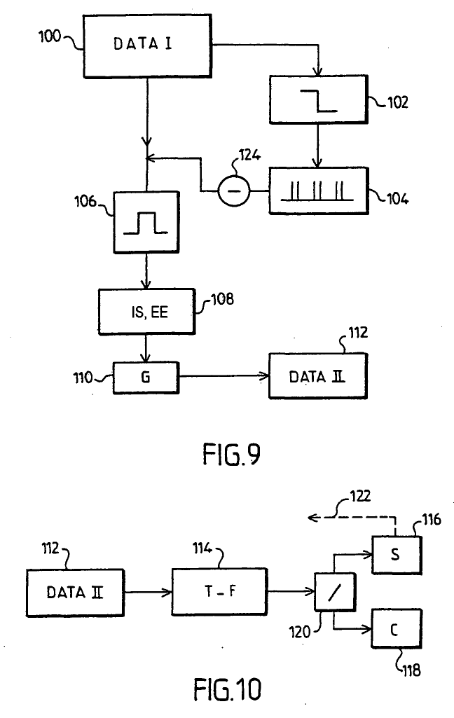

Referring to FIG. 9, the processing for the purposes of the present invention starts with the inputting of raw sampled data stored at step 100 (DATA I). Advantageously, provision is made at steps 102 and 104 to filter out the sounds from the heartbeats. For this purpose, a low-pass filter 102 lets through the very low frequencies (of the order of a few HZ), while an analyzer 104 identifies the frequency of the heartbeats and reconstructs the signal therefrom. This signal is subtracted from the raw data signal DATA I at step 124.

At step 106, the signal representative of the respiratory sound, containing as the case may be adventitious sounds (and without the sounds of the heartbeats) is filtered by a band-pass filter, preferably between 50 and 2 000 Hz.

At step 108, a signal detector adjusts the respiratory cycle with respect to a standard cycle, the duration of which is predetermined, especially according to the patient's characteristics (age, sex, etc.). Such an adjustment advantageously makes it possible to suppress changes of frequency that are due to the durations of inspirations and expirations, which are generally variable. Thus, an inspiration start IS and an expiration end EE, to which we will return later on, are detected.

At step 110, a dynamic range compressor/expander adjusts the gain G as a function of the local level, with a chosen time constant (preferably from 0.1 to 0.5 seconds). Advantageously, this time constant is relatively short, so as to enrich the spectra that will be obtained in subsequent processing. This function thus makes it possible to increase the dynamic range of small signals so as to facilitate qualitative analysis of the signal. It also makes it possible to obtain a “zoom” effect on low-amplitude passages in the measured signals. On the other hand, if the time constant is chosen to be higher, a spectral signal will be obtained whose contrast is higher. Thus, in a first analysis, a relatively short time constant may be chosen so as to obtain a rich spectrum, whereas in a second processing step (especially when the types of adventitious sound have been identified) a longer time constant may be chosen so as to obtain only predominant signals representative of a particular adventitious sound in the spectra obtained.

After the step of modifying the gain G, data DATA II is recovered at step 112, this data thus being preprocessed.

Referring to FIG. 10, this preprocessed data is stored in the memory of the device, for the purpose of performing a time-frequency transform of the type described in the Applicant's application WO 00/00736. After this conversion step 114, the device includes, in an advantageous embodiment, a module 120 for automatically recognizing adventitious sounds that are likely to be identified in the spectra obtained after step 114. Preferably, the module 120 is in the form of a computer program that is stored in the read-only memory of the device and can be executed by the processor for the purpose of identifying the intensity maxima:
as a function of time, and at a constant frequency, in order to identify crackling (coarse crackles or fine crackles); and
as a function of frequency, and at a constant time, for the purpose of identifying sibilant rhonchi.

Thus, the module 120 performs successive frequency scans in order to identify the sibilant rhonchi and time scans in order to identify the crackles.

In a less sophisticated variant, the spectra obtained at step 114 may be displayed on the screen of the device, while a manipulator, by displaying the spectra on the screen, determines whether adventitious sounds relate to sibilant rhonchi or to crackles.

The processing continues at step 116, when the spectra obtained indicates the presence of sibilant rhonchi. Preferably, at step 116, the preprocessing steps of FIG. 9 are repeated (arrow 122) on fresh respiratory sound data, if necessary using a longer time constant than in step 110, this being done, so as to average the spectra indicating the presence of sibilant rhonchi, for example in an average of five spectra. This averaging performed at step 116 thus makes it possible to smooth out the spectra obtained and to identify frequency ranges within which the measured sibilant rhonchi change.

Of course, in a less sophisticated variant, the sibilant rhonchi may be identified in a single spectra obtained in step 114. However, the average of the spectrograms is used to attenuate the non-steady-state phenomena, whereas the steady-state phenomena, (such as sibilant rhonchi) are confirmed.

Moreover, at step 118 and when crackling (coarse crackles or fine crackles) are identified, the preprocessing operations of FIG. 9 may be repeated, for example just once, with a higher time constant, so as to contrast the spectrum obtained at step 114, where appropriate. In this case, it will be attempted to quantify the number of non-steady-state phenomena observed, for example the number of crackles, their height in terms of frequency, their width in time, their intensity, etc.

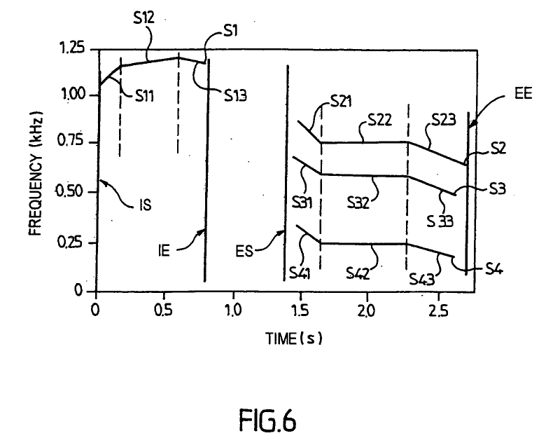

FIG. 6 represents the modeling of the sibilant rhonchi that appear in the spectrum of FIG. 3. When several spectra are averaged at step 116, the non-steady-state phenomena are filtered out, while only the sibilant rhonchi are retained. These sibilant rhonchi are manually “linearized” using a conventional graphical processing operation.

In a more sophisticated variant, statistical processing establishes, as a function of time, linear regressions of the frequency variations of these sibilant rhonchi, preferably within six temporal ranges, as will be seen below.

Advantageously, at step 108, it is also possible to determine, by determining an inspiration start instant IS and an expiration end instant EE, an inspiration end instant IE and an expiration start instant ES since, referring to FIG. 3, the intensity of the signals detected between IE and ES show a sudden drop.

In each temporal range IS-IE and ES-EE, the aforementioned statistical processing (for example, a computer program capable of statistically managing the time/frequency data calculated at step 114) establishes a first linear regression S11, S21, S31, S41, and a threshold value that is a function of the standard deviation in this regression (for example, a multiple of the standard deviation). When the statistical processing detects a plurality of successive points that lie significantly outside the regression (depending on the aforementioned threshold value), a second linear regression S12, S22, S32, S42 is calculated. Again, associated with this linear regression is a threshold value that depends on the standard deviation in this linear regression. The processing continues by calculating a third linear regression S13, S23, S33 and S43, when successive points lie significantly outside the previous linear regression.

Of course, the aforementioned threshold values may be different depending on the first, second or third linear regressions. Typically, the practitioner knows that, at the start of an inspiration or an expiration (S11, S21, S31, S41), the frequency of a sibilant rhonchus varies suddenly with time. This frequency then becomes relatively stable over time (plateaus S12, S22, S32 and S42). At the end of an inspiration or an expiration (S13, S23, S33, S43), this frequency again varies suddenly with time (generally decreases rapidly).

Advantageously, the statistical processing makes it possible to identify these three phases for the various sibilant rhonchi detected. In the example shown in FIG. 6, the inspiration phase exhibits a single sibilant rhonchus, with three clear inspiration start, frequency-stabilization and inspiration end phases (with a decrease in the frequency of the sibilant rhonchus). As regards the expiration phase, this exhibits three sibilant rhonchi, with frequency reductions initially, then a frequency stabilization and frequency reductions at the end of expiration.

More precisely, the expiration phase exhibits a single polyphonic sibilant rhonchus, with a sibilant rhonchus at the fundamental frequency S4 and two sibilant rhonchi at harmonic frequencies S2 and S3.

A sibilant rhonchus may conventionally be decomposed into a Fourier series of the type:
S(t)=sin(ω₀t)+ΣA_{I} sin(iω₀t).

In this expression, ω₀ is a function of time. In the example shown in FIG. 6, this function is linear since the modeling of the frequency variation of the sibilant rhonchi is preferentially linear in the example, with substantially linear frequency increase or decrease phases and phases in which these frequencies are approximately constant.

The processing by the device according to the invention advantageously evaluates the coefficients of the linear variation of the frequency in each inspiration or expiration phase of the sibilant rhonchi. These linear variation parameters are important data for diagnosis by the practitioner in terms of the patient's pathology, as will be seen below.

During the relatively frequency-stable phases Si2, preferential provision is made to average the respective frequencies of the sibilant rhonchi for the purpose of storing these average frequencies, while the frequencies of the other phases Si1 and Si2 are parameterized in a linear variation model.

The number of sibilant rhonchi having the lowest fundamental frequencies (S1 and S4 in the example shown) is stored as parameter representing the adventitious sound.

Another important parameter is the number of harmonics that a polyphonic sibilant rhonchus has, and the ratio of their intensities (the mean value of each parameter Aᵢ of the Fourier series). The processing by the device according to the invention indicates the numerical values of these parameters, for example by displaying them on a display screen of the device.

Provision is also made to store these parameters in a read-only memory of the device, for the purpose of constructing a database. On the basis of these parameters, the soundcard 1 can cooperate with the processor and this read-only memory for the purpose of reconstructing an acoustic signal that comprises the parameters calculated and stored in the database in the manner described above.

For example, provision may be made to store sampled data of a normal vesicular murmur, on which is superposed a sound whose parameters are taken from the aforementioned database.

When the parameters are stored in the read-only memory ROM of the device, this memory being arranged and structured according to the aforementioned database, a set of stored parameters is representative of an associated pathology, and more particularly of a stage, known by the practitioner, in the development of this pathology.

Reference will now be made to FIGS. 7 and 8 in order to describe the modeling of the crackling (fine crackles in FIG. 7 and coarse crackles in FIG. 8).

A processing module is provided, in the device, that is capable at the end of step of 118 of identifying the frequency maxima in the measured intensities for each crackle and an average width L of the crackle, which corresponds to a temporal range within which this crackle lies. Thus, each crackle is modeled according to its frequency height H, relative to the vesicular murmur (background noise at about 300 Hz), and by the temporal range L within which this crackle lies and which allows it to be characterized in terms of “coarse crackles” or “fine crackles”.

Advantageously, the number of crackles is detected manually or automatically.

The practitioner may, here again, make a diagnosis on the basis of these data (number of crackles in a respiratory cycle, average width L of the crackles and frequency height).

In addition, the parameters of these crackles (width L and height H) may be stored and classified in a database as “fine crackles” or “coarse crackles”.

Preferably, in the rectangles that model the crackles shown in FIGS. 7 and 8, an average intensity is assigned to each crackle and this average value is stored with the parameters L and H in the database.

Conversely, using the parameters stored in this database, it is possible to recreate a respiratory sound comprising adventitious sounds of the “crackle” type (parameterized according to their width, their height and their number) that are superposed on a normal vesicular murmur and, where appropriate, on modeled sibilant rhonchi, if it is desired to listen to a complex respiratory sound.

Of course, another particularly advantageous application consists in operating the device for a patient at home, when the parameters extracted in the manner described above from the measured respiratory sounds (frequency variation of the sibilant rhonchi, ratio of the intensity of the harmonics, width, height and number of crackles) can be transmitted via a communication link in an Internet-type network to a diagnosis unit. The diagnosis unit is therefore equipped with a processing module that recomposes the sound thus parameterized, while the practitioner listens to this sound in order to make his diagnosis.

In this embodiment, the transmission via the communication link is rapid since the transmitted data (adventitious sound parameters) are much less than unprocessed sound data that would have been transmitted for the practitioner to listen to.

In a variant, the device may compare the measured parameters with parameters stored in a database that the memory of the device has, while an alarm module (for example an acoustic signal or a visual signal displayed on the screen) is activated if this comparison reveals an advanced stage of the pathology, corresponding as the case may be to a crisis.

The database may be arranged as a shared reference system covering a large number of respiratory sounds, these being progressively added to, in particular under the control of specialists and/or experts.

Of course, the present invention is not limited to the embodiment described above by way of example, rather it extends to other variants that nevertheless remain defined within the context of the claims appended hereto.

1. A device for assistance in the analysis of respiratory sounds, of the type comprising a working memory (2), capable of storing samples of a signal representing a respiratory sound, and a computing module (4, 3) capable of cooperating with the working memory in order to carry out a time and frequency conversion of the signal (114), characterized in that it furthermore includes means (4, 3, 120) for identifying, in a converted signal, at least one component relating to an adventitious sound (Si; FCi; Cci) and in that the computing module is designed to extract a set of parameters (Ai, ω₀; H, L) characterizing this adventitious sound from the converted signal.

2. The device as claimed in claim 1, characterized in that said means are designed to identify the component that represents the adventitious sound in a frequency and time representation (FIG. 3, FIG. 4, FIG. 5) of the converted signal.

3. The device as claimed in claim 2, characterized in that it includes an acquisition member (92, 93), whereas the means for identifying the component are manual.

4. The device as claimed in either of claims 1 or 2, characterized in that the computing module is furthermore capable of comparing (120) successive intensity values of the converted signal in order to identify said component.

5. The device as claimed in claim 4, characterized in that the computing module (120) is designed to compare the intensity values by carrying out:
a frequency scan of the intensities, for various successive instants, when the adventitious sound to be identified is a set of sibilant rhonchi; and/or
a time scan of the intensities, for various frequencies, when the adventitious sound to be identified is a set of crackles.

6. The device as claimed in claim 1, characterized in that the parameters characterizing a set of crackles comprise the number of crackles identified, and also a temporal segment (L) and preferably a mean intensity in this temporal segment, for each crackle.

7. The device as claimed in claim 6, characterized in that the parameters furthermore include a frequency range (H) within which each identified crackle lies.

8. The device as claimed in claim 1, characterized in that the parameters characterizing a set of sibilant rhonchi comprise, for each sibilant rhonchus, a mean frequency over a stable phase of the sibilant rhonchus (Si2) and at least one coefficient of linear variation in frequency of the start (Sil) and/or of the end (Si3) of the sibilant rhonchus.

9. The device as claimed in claim 1, characterized in that the parameters characterizing a set of sibilant rhonchi comprise the number of fundamental sibilant rhonchi of lowest frequencies (S1, S4).

10. The device as claimed in claim 1, characterized in that the parameters characterizing a set of sibilant rhonchi comprise, for each fundamental sibilant rhonchus (S4) and its harmonic sibilant rhonchi (S2, S3), the number of harmonic sibilant rhonchi and a mean ratio (Ai) of the intensities of the harmonics to the intensities of the fundamental.

11. The device as claimed in claim 1, characterized in that the computing module is furthermore designed to compare said parameters with values prerecorded in an ordered memory (ROM), comprising data of sets of parameters, each set characterizing at least one adventitious sound, and in that the device furthermore includes a warning module suitable for being activated or not, depending on the comparison.

12. The device as claimed in claim 1, characterized in that the parameters characterizing an adventitious sound are stored in succession in an ordered memory (4).

13. The device as claimed in either of claims 11 or 12, characterized in that the ordered memory (4) is structured as a database.

14. The device as claimed in claim 13, characterized in that it furthermore includes a telecommunication module (MODEM) for remote consultation of the database.

15. A removable medium intended to cooperate with a reader (5) of a device as claimed in claim 1, characterized in that it includes data for programs for using the computing module.

16. The removable medium as claimed in claim 14, characterized in that it furthermore includes prerecorded data of parameters (Ai, ω₀; H, L) characterizing adventitious sounds.
